(19) United States
(12) Patent Application Publication
(10) Pub. No.: US 2025/0336486 A1
(43) Pub. Date: Oct. 30, 2025

(54) CHEMICAL SEARCH QUERY USING VECTOR DATABASE

(71) Applicant: Microsoft Technology Licensing, LLC, Redmond, WA (US)

(72) Inventor: Chi CHEN, Renton, WA (US)

(73) Assignee: Microsoft Technology Licensing, LLC, Redmond, WA (US)

(21) Appl. No.: 18/646,523

(22) Filed: Apr. 25, 2024

(51) Int. Cl. G16C 20/70; G06F 16/2455; G16C 20/90

(52) U.S. Cl. CPC G16C 20/70; G06F 16/24558; G16C 20/90

(57) ABSTRACT

Examples are disclosed that relate to forming embeddings comprising vector representations of chemical structures, and performing chemical similarity searches based on embeddings. One example provides a method comprising receiving a query comprising chemical structure information for a target chemical object, and, based on the query, inputting the chemical structure information into a trained neural network configured to form embeddings of chemical structures. The method further comprises receiving an embedding for the target chemical object from the trained neural network, the embedding comprising a vector representation of the target chemical object, based at least on a similarity score between the embedding for the target chemical object and each of one or more embeddings stored in a vector database, retrieving query results from the vector database, the query results comprising a set of embeddings and metadata for a corresponding set of chemical objects, and outputting the query results.

### BACKGROUND

A chemical information database can be used to help search for chemicals that are structurally similar to a selected chemical of interest. Search methods often use structural comparisons that utilize rule-based algorithms.

### SUMMARY

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter. Furthermore, the claimed subject matter is not limited to implementations that solve any or all disadvantages noted in any part of this disclosure.

Examples are disclosed that relate to forming embeddings comprising vector representations of chemical structures, and performing chemical similarity searches based on embeddings. One example provides a method of forming a vector database using a neural network. The method comprises inputting a set of training data into the neural network, the set of training data comprising structural information for each chemical object of a plurality of chemical objects. The method further comprises training the neural network, and using the trained neural network to form the vector database by generating embeddings of chemical structures, each embedding comprising a vector representation of a chemical structure.

Another example provides a method, enacted on a computing system, for generating a vector database for performing a chemical similarity search. The method comprises inputting a database of chemical objects into a trained neural network to generate embeddings, each embedding comprising a vector representation of a chemical object of the database. The method further comprises saving the embeddings with metadata in a vector database, the metadata associated with an embedding comprising an identification of a chemical object corresponding to the embedding.

Another example provides a method enacted on a computing system. The method comprises receiving a query comprising chemical structure information for a target chemical object, and, based on the query, inputting the chemical structure information into a trained neural network configured to form embeddings of chemical structures. The method further comprises receiving an embedding for the target chemical object from the trained neural network, the embedding comprising a vector representation of the target chemical object, based at least on a similarity score between the embedding for the target chemical object and each of one or more embeddings stored in a vector database, retrieving query results from the vector database, the query results comprising a set of embeddings and metadata for a corresponding set of chemical objects, and outputting the query results.

### BRIEF DESCRIPTION OF THE DRAWINGS

FIG. 1 schematically shows an example of a computing architecture for implementing a searchable vector database.

FIG. 2 shows a flow diagram for an example method of processing a chemical search query.

FIG. 3 shows a flow diagram for an example method of training a neural network to learn embeddings and forming a vector database comprising embeddings.

FIG. 4 shows a block diagram of an example computing system.

### DETAILED DESCRIPTION

In the field of cheminformatics and materials science, it can be challenging to compare chemicals, or to identify materials with a desired property. Chemical information databases can be used to help search for chemicals that are structurally similar to a target chemical. However, search methods often use simplistic structural comparisons that utilize rule-based algorithms or fingerprint methods. Such structural comparisons can fail to account for nuanced similarities and/or functional relationships. Search algorithms can include additional rules to attempt to capture a greater number of functional relationships. However, more complicated algorithms can be slow.

Accordingly, examples are disclosed that relate to using deep learning models to generate embeddings for chemical objects, including molecules and materials. Embeddings can then be used to form a vector database for use in a chemical similarity search. Briefly, a neural network is trained using training data comprising chemical structure data. Training can be supervised or unsupervised, as described in more detail below. During training, one or more embedding layers learn to generate latent representations, or embeddings, of chemical structures. After training, the neural network can be used to generate embeddings of chemical objects.

Embeddings can capture intricate properties and relationships of chemical structures in a multidimensional space. To generate an embedding, a chemical structure is input into the trained neural network, and the embedding for the chemical structure is output by the embedding layer. The embedding comprises a vector representation of the chemical structure, wherein the vector representation is formed from coefficients output by the embedding layer of the trained neural network. The generated embeddings can be stored in a vector database. Embeddings are stored with metadata comprising information identifying the chemical objects corresponding to the embeddings. The vector database can then be used for performing a chemical similarity search.

To query the vector database, chemical structure information for a target chemical object is input into the trained neural network. The chemical structure information can include, e.g., cartesian coordinates of atoms in a chemical object and/or bonding information for a chemical object. Examples of inputs of chemical structure information include text strings (e.g., a chemical name or a simplified molecular-input line-entry system (SMILES) string) and 3-dimensional (3D) structural data (e.g., XYZ format, crystallographic information file (CIF) format, MOL format, Protein Data Bank (PDB) format, etc.). The trained neural network generates an embedding comprising a vector representation of the target chemical object. The embedding is used to query the vector database to identify other chemical objects that are structurally similar to the target chemical object. As described in more detail below, the structural similarity of two chemical objects can be quantified based on the respective embeddings for the chemical objects. In this manner, one or more chemical objects that are structurally similar to the target chemical object can be identified and output to a user.

A vector database can offer improvements over traditional cheminformatics databases that are not optimized for high-dimensional data produced by deep-learning models. By using a vector database rather than a rule-based search method, querying can be more efficient than other methods. Additionally, the vector database can be configured for storing embeddings and high-efficiency querying of embeddings. In some examples, 100 million entries can be searched in less than 0.1 seconds. Further, by using deep learning for embeddings, the system can capture complex molecular characteristics that other methods (e.g., rule-based algorithms or fingerprint methods) can overlook. This helps provide chemical similarity searches with greater accuracy and more meaningful results than other search methods. This can be useful in various applications, such as drug discovery, material design, and environmental studies.

Examples are also disclosed that relate to fine-tuning the trained neural network. After training the neural network to generate embeddings, the neural network can be fine-tuned using additional training data. The additional training data can comprise, e.g., a chemical or physical property to be learned by the neural network. After fine-tuning, the neural network can be used to predict the property. Then, a further query to the vector database can include property information. For example, query results can be weighted or filtered based on a predicted property for each entry in the vector database.

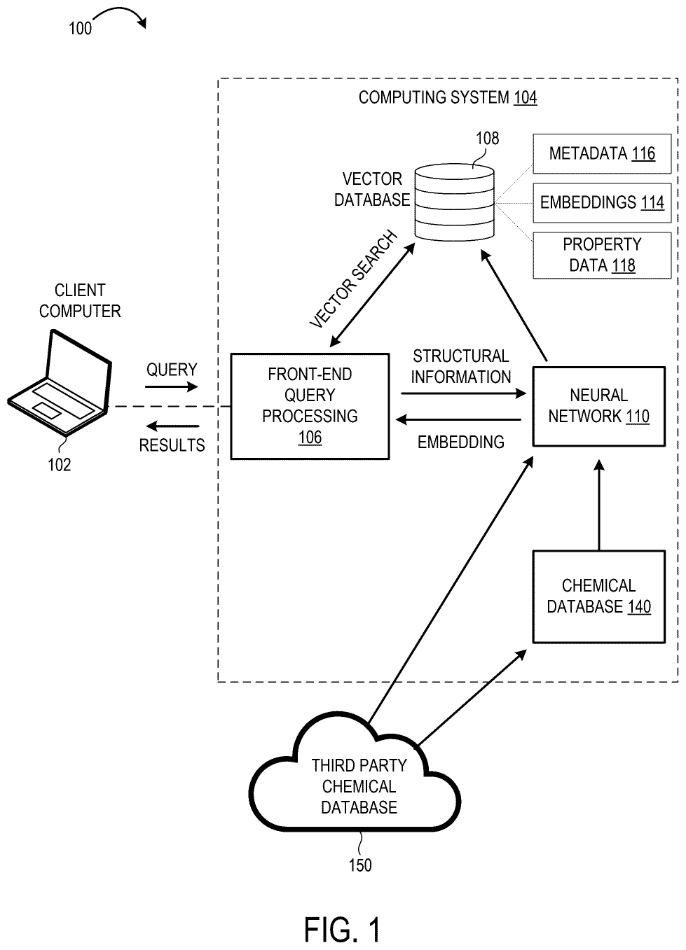

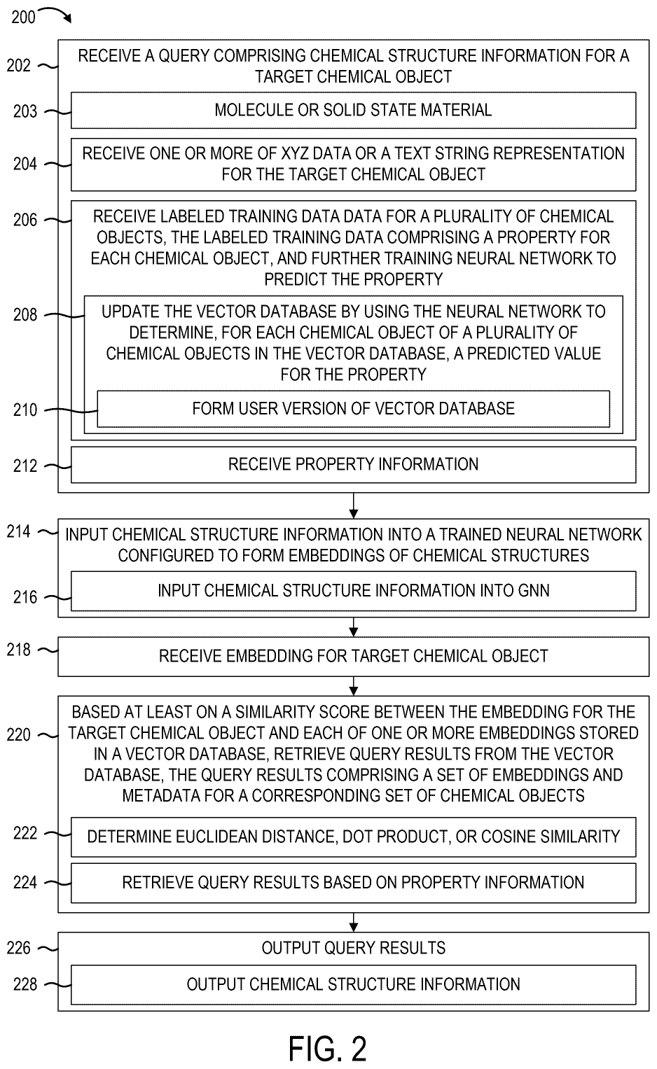

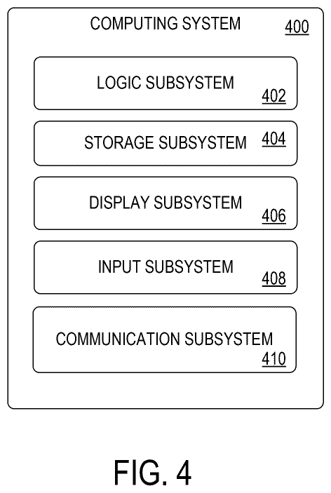

FIG. 1 schematically shows an example of a computing architecture 100 for processing a chemical search query. As illustrated in FIG. 1, a client computer 102 can submit a query to a computing system 104. Computing system 104 comprises one or more processors configured to process queries and perform various other functions. An example method of processing queries is discussed below with regard to FIG. 2. Computing system 104 can represent a data center in some examples. Examples of computing systems are described in more detail below with regard to FIG. 4.

Computing system 104 comprises a front-end module 106 for query processing. Computing system 104 further comprises a storage system storing data for a vector database 108, one or more neural networks 110, and training data 112. Vector database 108 comprises a plurality of embeddings 114 for a respective plurality of chemical objects, including molecules and/or materials (e.g., crystal structures or other representation of solids). Each embedding comprises a vector representation of the structure of a chemical object. The vector database further comprises metadata 116. Metadata 116 can comprise any suitable information for chemical objects corresponding to embeddings 114. In some examples, metadata 116 can include, for each embedding, a chemical object identification (ID) corresponding to the embedding. Examples of metadata include chemical names, chemical formulas, and ID numbers. In some examples, vector database 108 further comprises property data 118 for one or more chemical objects corresponding to embeddings 114. In some examples, the property data comprises an energy for a chemical object, such as a formation energy. Further examples of properties include a boiling point, an ionization energy, a bandgap, and classifications, such as metal vs. non-metal or conducting vs. insulating. Further, property data 118 can comprise experimental properties and/or predicted properties. In some examples property data 118 comprises properties predicted using a neural network, such as neural network 110. In some such examples, the predicted properties are generated during a fine-tuning process, as discussed in more detail below.

Neural network 110 is configured to generate embeddings for chemical objects. Examples of neural networks include transformers, graph-transformers, convolutional neural networks (CNNs), and/or graph neural networks (GNNs). GNNs are trained to perform inference on data described by a mathematical graph. Graphs can be a suitable choice for representing a chemical object, such as a molecule or solid state material (e.g. a unit cell of a crystal), where nodes represent atoms and edges represent bonds. Examples of GNNs suitable for generating embeddings include GNNs trained using the Graphormer deep learning package (Ying, Chengxuan, et al. “Do transformers really perform badly for graph representation?.” Advances in neural information processing systems 34 (2021): 28877-28888). Any suitable configuration can be used for neural network 110. In some examples, neural network 110 comprises a GNN configured with ≥6 layers. In some examples, neural network 110 comprises ≥80 hidden dimensions. In some more specific examples, neural network 110 comprises 12 layers, 32 attention heads, and 24 hidden dimensions for each attention head. In other examples, any other suitable configuration can be used.

Neural network 110 can be trained using any suitable training data. Computing system 104 optionally comprises a chemical database 140 that can be used to provide training data. Additionally or alternatively, a third party chemical database 150 can be used to provide training data for training neural network 110. One example of a publicly accessible third party chemical database is PubChem, available at pubchem.ncbi.nlm.nih.gov (Kim S, Chen J, Cheng T, et al. PubChem 2023 update. Nucleic Acids Res. 2023; 51 (D1): D1373-D1380).

Neural network 110 can be trained using supervised training or unsupervised training. In supervised training, labeled training data is input into neural network 110 and the neural network is trained to predict a property, such as an energy. In some examples, a computationally inexpensive energy calculation can be used to predict energies for use in the supervised training process. In other examples, any other suitable predicted property can be used. In further example, empirical data can be used.

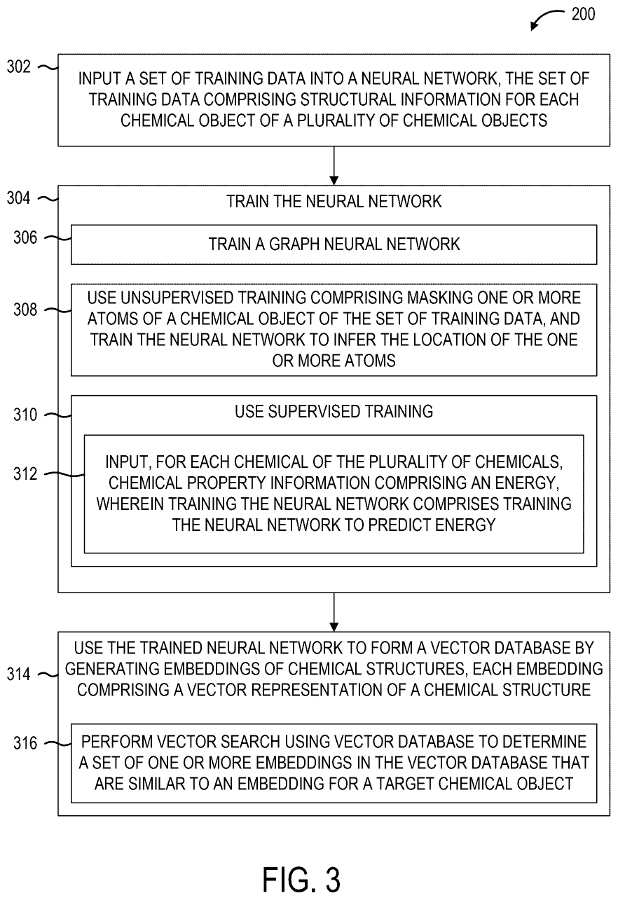

Alternatively, neural network 110 can be trained using unsupervised training. In unsupervised training, the neural network can be trained to predict structural information of a chemical object. For example, one or more atoms of a chemical object can be masked, and the neural network learns to predict a type and/or location of the masked atom in the chemical object. Examples for training neural network 110 are discussed below with regard to FIG. 3.

After training, neural network 110 can be used to generate embeddings based on chemical structures. As discussed above, an embedding comprises a vector representation of a chemical structure. In some examples, the length of the vector corresponds to the number of hidden dimensions in the neural network. Embeddings can be used to form vector database 108. For example, a plurality of chemical objects from chemical database 140 and/or third party chemical database 150 can be input into neural network 110 to generate embeddings. In some examples, a chemical database can be supplied by a user (e.g., from client computer 102). The generated embeddings are stored in vector database 108 as embeddings 114. Each embedding can be stored with metadata 116, such as a chemical object ID corresponding to the embedding.

Neural network 110 can be further trained using additional training data comprising new chemical objects. In contrast to static rule-based algorithms, computing system 104 can continuously learn and update neural network 110 to understand additional chemical structures. After the further training, neural network 110 can be used to update embeddings 114. This dynamic learning aspect allows vector database 108 to adapt to new data and discoveries.

Additionally, after initial training, neural network 110 can be fine-tuned. A fine-tuning process can include further training neural network 110 to predict a property, for example, to be stored in vector database 108 in property data 118. In some examples, fine-tuning can be performed using user data. For example, client computer 102 can transmit additional training data to computing system 104 to be used in the fine-tuning process. In some such examples, the fine-tuning process can be used to form a user version of vector database 108. This can allow a user to form a customized vector database without affecting a public version of vector database 108.

Further, neural network 110 can be used to generate an embedding that can be used in a chemical search query. For example, in response to a query from client computer 102 received at front-end module 106, computing system 104 inputs structural information for a target chemical object into neural network 110. Neural network 110 generates an embedding comprising a vector representation of the target chemical object. The embedding can be output to front-end module 106. Computing system 104 can then perform a vector search of vector database 108 to retrieve embeddings similar to the embedding for the target chemical object. The vector search can employ a similarity score, for example. Any suitable similarity score can be used, such as a Euclidean distance, a Hamming distance, a dot product, or a cosine similarity. A relatively high degree of similarity between two embeddings indicates a relatively high degree of topological similarity between the two chemical objects corresponding to the embeddings. Thus, based on the embedding of the target chemical object and a similarity score metric, the vector search can retrieve a set of K similar chemical objects from the vector database. In some examples, a chemical search query can include property information. As discussed above, query results can be weighted and/or filtered based on the property information specified in the query and property data 118 for corresponding chemical objects and embeddings 114. Embeddings can capture complex molecular characteristics and intricate relationships between chemicals in a multidimensional space. In this manner, embeddings can be more nuanced than other solutions that utilize rule-based algorithms or fingerprint methods for structural comparisons between different chemical structures. As such, a vector search of vector database 108 can provide a chemical similarity search with greater accuracy and more meaningful results than other search methods.

FIG. 2 shows a flow diagram for an example method 200 for processing a chemical search query using a vector database (e.g., vector database 108) and a neural network (e.g., neural network 110). Computing architecture 100 is an example of a computing architecture for processing chemical search queries using method 200.

Method 200 comprises, at 202, receiving a query comprising chemical structure information for a target chemical object. In some examples, at 203, the chemical structure information for the target chemical object comprises structural information for a molecule or structural information for a solid state material. The query can comprise any suitable chemical structure information. In some examples, at 204, method 200 comprises receiving one or more of XYZ data for the target chemical object, or a text string representation of the target chemical object. XYZ data can comprise cartesian coordinates of atoms in a chemical object using any suitable format. Examples of XYZ data includes chemical structure information in an XYZ format, CIF format, MOL format, and PDB format. A text string representation can comprise any suitable information identifying a chemical object or describing bonding within a chemical object. Examples of text string representations for a chemical object include a chemical name, a chemical formula, and a SMILES string.

In some examples, method 200 comprises fine-tuning the neural network prior to receiving the query at 202. For example, at 206, method 200 optionally comprises receiving labeled training data for a plurality of chemical objects, the labeled training data comprising a property for each chemical object, and further training the neural network to predict the property. In some examples the size of the labeled training data can be relatively small compared to the size of the training data initially used to train the neural network and generate embeddings. In some examples, at 208, method 200 comprises, after further training the neural network, updating the vector database by using the neural network to determine, for each chemical object of a plurality of chemical objects in the vector database, a predicted value for the property. In some examples, at 210, updating the vector database at 208 comprises forming a user version of the vector database. This can allow a user of a public system to fine-tune the vector database without affecting a public version of the vector database for other users. In some examples, at 212, receiving the query comprises receiving property information. Property information can be used, e.g., to filter query results or refine a chemical similarity search based on predicted property values generated at 208.

Method 200 further comprises, at 214, inputting the chemical structure information into a trained neural network configured to form embeddings of chemical structures. Any suitable neural network can be used. In some examples, at 216, the chemical structure information is input into a GNN. Neural network 110 is an example of a neural network that can be used at step 208.

Method 200 further comprises, at 218, receiving an embedding for the target chemical object from the trained neural network, the embedding comprising a vector representation of the target chemical object.

Continuing, at 220, method 200 further comprises, based at least on a similarity score between the embedding for the target chemical object and each of one or more embeddings stored in a vector database, retrieving query results from the vector database. The query results comprise a set of embeddings and metadata for a corresponding set of chemical objects. The metadata can comprise, e.g., chemical object IDs for the corresponding set of chemical objects. Any suitable method can be used to determine a similarity score at step 220. In some examples, at 222, retrieving the query results comprises determining one or more of a distance, a dot product, or a cosine similarity for the embedding for the target chemical object and an embedding stored in the vector database. In some examples, at 224, the method includes receiving property information at step 206, and retrieving query results from the vector database is further based on property information and the predicted values for the property for corresponding chemical objects of the query results.

Method 200 further comprises, at 226, outputting the query results. Referring to FIG. 1, computing system 104 can output query results to client computer 102. Any suitable information pertaining to the query results can be output, such as a chemical name or a chemical formula for each chemical object in the query results. In some examples, at 228, method 200 comprises, for each chemical object in the one or more chemical objects of the query results, outputting structural information for the chemical object.

FIG. 3 shows a flow diagram for an example method of training a neural network (e.g., neural network 110). At 302, method 300 comprises inputting a set of training data into a neural network, the set of training data comprising structural information for each chemical object of a plurality of chemical objects.

At 304, method 300 further comprises training the neural network. In some examples, at 306, training the neural network comprises training a graph neural network (GNN). As discussed above, a graph can be a suitable choice for representing a chemical object. In some examples, at 308, training the neural network comprises using unsupervised training, wherein the unsupervised training comprises masking one or more atoms of a chemical object of the set of training data, and training the neural network to predict the location of the one or more atoms. In some examples, at 310, training the neural network comprises using supervised training. Supervised training uses labeled training data. In some such examples, at 312, the method comprises inputting, for each chemical object of the plurality of chemical objects, chemical property information comprising an energy, and wherein training the neural network comprises training the neural network to predict energy.

Continuing, at 314, method 300 further comprises using the trained neural network to form a vector database by generating embeddings of chemical structures, each embedding comprising a vector representation of a chemical structure. The embeddings are stored in the vector database with metadata (e.g., IDs for the chemical objects associated with the embeddings). In some examples, at 316, method 300 further comprises performing a vector search using the vector database to determine a set of one or more embeddings in the vector database that are similar to an embedding for a target chemical object.

Thus, the disclosed examples provide for generating vector databases using a neural network, and performing a chemical similarity search using the vector database. By using deep learning for embeddings, the disclosed examples can capture complex molecular characteristics that other methods might overlook, leading to more accurate and meaningful similarity searches. Further, the trained neural network can form embeddings for a wide range of chemical structures. This makes it applicable in various fields, from drug discovery to materials engineering. By using a vector database rather than a rule-based search method, querying can be more efficient than other methods.

In some embodiments, the methods and processes described herein may be tied to a computing system of one or more computing devices. In particular, such methods and processes may be implemented as a computer-application program or service, an application-programming interface (API), a library, and/or other computer-program product.

FIG. 4 schematically shows a non-limiting embodiment of a computing system 400 that can enact one or more of the methods and processes described above. Computing system 400 is shown in simplified form. Computing system 400 may take the form of one or more personal computers, server computers, tablet computers, home-entertainment computers, network computing devices, gaming devices, mobile computing devices, mobile communication devices (e.g., smart phone), and/or other computing devices. Computing system 104 is an example of computing system 400.

Computing system 400 includes a logic subsystem 402 and a storage subsystem 404. Computing system 400 may optionally include a display subsystem 406, input subsystem 408, communication subsystem 410, and/or other components not shown in FIG. 4.

Logic subsystem 402 includes one or more physical devices configured to execute instructions. For example, the logic subsystem may be configured to execute instructions that are part of one or more applications, services, programs, routines, libraries, objects, components, data structures, or other logical constructs. Such instructions may be implemented to perform a task, implement a data type, transform the state of one or more components, achieve a technical effect, or otherwise arrive at a desired result.

The logic subsystem may include one or more processors configured to execute software instructions. Additionally or alternatively, the logic subsystem may include one or more hardware or firmware logic subsystems configured to execute hardware or firmware instructions. Processors of the logic subsystem may be single-core or multi-core, and the instructions executed thereon may be configured for sequential, parallel, and/or distributed processing. Individual components of the logic subsystem optionally may be distributed among two or more separate devices, which may be remotely located and/or configured for coordinated processing. Aspects of the logic subsystem may be virtualized and executed by remotely accessible, networked computing devices configured in a cloud-computing configuration.

Storage subsystem 404 includes one or more physical devices configured to hold instructions executable by the logic subsystem to implement the methods and processes described herein. When such methods and processes are implemented, the state of storage subsystem 404 may be transformed—e.g., to hold different data.

Storage subsystem 404 may include removable and/or built-in devices. Storage subsystem 404 may include optical memory (e.g., CD, DVD, HD-DVD, Blu-Ray Disc, etc.), semiconductor memory (e.g., RAM, EPROM, EEPROM, etc.), and/or magnetic memory (e.g., hard-disk drive, floppy-disk drive, tape drive, MRAM, etc.), among others. Storage subsystem 404 may include volatile, nonvolatile, dynamic, static, read/write, read-only, random-access, sequential-access, location-addressable, file-addressable, and/or content-addressable devices.

It will be appreciated that storage subsystem 404 includes one or more physical devices. However, aspects of the instructions described herein alternatively may be propagated by a communication medium (e.g., an electromagnetic signal, an optical signal, etc.) that is not held by a physical device for a finite duration.

Aspects of logic subsystem 402 and storage subsystem 404 may be integrated together into one or more hardware-logic components. Such hardware-logic components may include field-programmable gate arrays (FPGAs), program- and application-specific integrated circuits (PASIC/ASICs), program- and application-specific standard products (PSSP/ASSPs), system-on-a-chip (SOC), and complex programmable logic devices (CPLDs), for example.

The terms “module,” “program,” and “engine” may be used to describe an aspect of computing system 400 implemented to perform a particular function. In some cases, a module, program, or engine may be instantiated via logic subsystem 402 executing instructions held by storage subsystem 404. It will be understood that different modules, programs, and/or engines may be instantiated from the same application, service, code block, object, library, routine, API, function, etc. Likewise, the same module, program, and/or engine may be instantiated by different applications, services, code blocks, objects, routines, APIs, functions, etc. The terms “module,” “program,” and “engine” may encompass individual or groups of executable files, data files, libraries, drivers, scripts, database records, etc.

It will be appreciated that a “service”, as used herein, is an application program executable across multiple user sessions. A service may be available to one or more system components, programs, and/or other services. In some implementations, a service may run on one or more server-computing devices.

When included, display subsystem 406 may be used to present a visual representation of data held by storage subsystem 404. This visual representation may take the form of a graphical user interface (GUI). As the herein described methods and processes change the data held by the storage subsystem, and thus transform the state of the storage subsystem, the state of display subsystem 406 may likewise be transformed to visually represent changes in the underlying data. Display subsystem 406 may include one or more display devices utilizing virtually any type of technology. Such display devices may be combined with logic subsystem 402 and/or storage subsystem 404 in a shared enclosure, or such display devices may be peripheral display devices.

When included, input subsystem 408 may comprise or interface with one or more user-input devices such as a keyboard, mouse, touch screen, or game controller. In some embodiments, the input subsystem may comprise or interface with selected natural user input (NUI) componentry. Such componentry may be integrated or peripheral, and the transduction and/or processing of input actions may be handled on- or off-board. Example NUI componentry may include a microphone for speech and/or voice recognition; an infrared, color, stereoscopic, and/or depth camera for machine vision and/or gesture recognition.

When included, communication subsystem 410 may be configured to communicatively couple computing system 400 with one or more other computing devices. Communication subsystem 410 may include wired and/or wireless communication devices compatible with one or more different communication protocols. As non-limiting examples, the communication subsystem may be configured for communication via a wireless telephone network, or a wired or wireless local- or wide-area network. In some embodiments, the communication subsystem may allow computing system 400 to send and/or receive messages to and/or from other devices via a network such as the Internet.

Another example provides a method enacted on a computing system. The method comprises receiving a query comprising chemical structure information for a target chemical object, based on the query, inputting the chemical structure information into a trained neural network configured to form embeddings of chemical structures, and receiving an embedding for the target chemical object from the trained neural network, the embedding comprising a vector representation of the target chemical object. The method further comprises, based at least on a similarity score between the embedding for the target chemical object and each of one or more embeddings stored in a vector database, retrieving query results from the vector database, the query results comprising a set of embeddings and metadata for a corresponding set of chemical objects, and outputting the query results. In some such examples, the method further comprises, for each chemical object in the corresponding set of chemical objects of the query results, outputting structural information for the chemical object with the query results. Additionally or alternatively, in some such examples, retrieving the query results from the vector database comprises determining one or more of a distance, a dot product, or a cosine similarity for the embedding for the target chemical object and an embedding stored in the vector database. Additionally or alternatively, in some such examples, inputting the chemical structure information into the trained neural network comprises inputting the chemical structure information into a graph neural network (GNN). Additionally or alternatively, in some such examples, receiving the query comprising the chemical structure information comprises receiving one or more of XYZ data for the target chemical object or a text string representation of the target chemical object. Additionally or alternatively, in some such examples, the method further comprises receiving labeled training data for a plurality of chemical objects, the labeled training data comprising a property for each chemical object of the plurality of chemical objects, and further training the neural network to predict the property. Additionally or alternatively, in some such examples, the method further comprises, after further training the neural network, updating the vector database by using the neural network to determine, for each chemical object of a plurality of chemical objects in the vector database, a predicted value for the property. Additionally or alternatively, in some such examples, updating the vector database is performed prior to the receiving the query, receiving the query comprises receiving property information, and retrieving query results from the vector database is further based on the property information and the predicted values for the property for corresponding chemical objects of the query results. Additionally or alternatively, in some such examples, updating the vector database comprises forming a user version of the vector database. Additionally or alternatively, in some such examples, the chemical structure information for the target chemical object comprises one or more of structural information for a molecule or structural information for a solid state material.

Another example provides a method of forming a vector database using a neural network. The method comprises inputting a set of training data into the neural network, the set of training data comprising structural information for each chemical object of a plurality of chemical objects, training the neural network, and using the trained neural network to form the vector database by generating embeddings of chemical structures, each embedding comprising a vector representation of a chemical structure, and storing the embeddings with metadata in the vector database. In some such examples, training the neural network comprises using unsupervised training, wherein the unsupervised training comprises masking one or more atoms in a molecule of a training data set and training the neural network to predict the location of the one or more atoms. Additionally or alternatively, in some such examples, training the neural network comprises using supervised training. Additionally or alternatively, in some such examples, the supervised training comprises inputting, for each chemical object of the plurality of chemical objects, chemical property information comprising an energy, and wherein training the neural network comprises training the neural network to predict energy. Additionally or alternatively, in some such examples, training the neural network comprises training a graph neural network.

Another example provides a computing system implementing a neural network. The computing system comprises a logic subsystem and a storage subsystem. The storage system comprises a vector database comprising a plurality of embeddings of a corresponding plurality of chemical objects, each embedding comprising a vector representation of a chemical structure for a corresponding chemical object, and metadata comprising chemical object identifications corresponding to the plurality of embeddings. The storage system further comprises instructions executable by the logic subsystem to receive a query comprising chemical structure information for a target chemical object, based on the query, input the chemical structure information into the neural network, the neural network configured to produce embeddings of chemical structures, receive, from the trained neural network, an embedding comprising a vector representation of the target chemical object, based on the vector representation of the target chemical object, retrieve query results from the vector database, the query results comprising a set of embeddings of chemical objects, and output the query results. In some such examples, the neural network comprises a graph neural network (GNN). Additionally or alternatively, in some such examples, the instructions executable to receive the query comprise instructions executable to receive one or more of XYZ data for the target chemical object or a text string representation of the target chemical object. Additionally or alternatively, in some such examples, the instructions are further executable to receive labeled training data for a set of chemical objects, the labeled training data comprising property information for a property for each chemical object of the set of chemical objects, and train the neural network to predict the property. Additionally or alternatively, in some such examples, the instructions are further executable to, after training the neural network, update the vector database by using the neural network to, for each chemical object of the plurality of chemical objects in the vector database, determine a predicted property value for the chemical object, and store the predicted property value in the vector database.

It will be understood that the configurations and/or approaches described herein are exemplary in nature, and that these specific embodiments or examples are not to be considered in a limiting sense, because numerous variations are possible. The specific routines or methods described herein may represent one or more of any number of processing strategies. As such, various acts illustrated and/or described may be performed in the sequence illustrated and/or described, in other sequences, in parallel, or omitted. Likewise, the order of the above-described processes may be changed.

The subject matter of the present disclosure includes all novel and non-obvious combinations and sub-combinations of the various processes, systems and configurations, and other features, functions, acts, and/or properties disclosed herein, as well as any and all equivalents thereof.

1. A method enacted on a computing system, the method comprising:
receiving a query comprising chemical structure information for a target chemical object;
based on the query, inputting the chemical structure information into a trained neural network configured to form embeddings of chemical structures;
receiving an embedding for the target chemical object from the trained neural network, the embedding comprising a vector representation of the target chemical object;
based at least on a similarity score between the embedding for the target chemical object and each of one or more embeddings stored in a vector database, retrieving query results from the vector database, the query results comprising a set of embeddings and metadata for a corresponding set of chemical objects; and
outputting the query results.

2. The method of claim 1, further comprising, for each chemical object in the corresponding set of chemical objects of the query results, outputting structural information for the chemical object with the query results.

3. The method of claim 1, wherein retrieving the query results from the vector database comprises determining one or more of a distance, a dot product, or a cosine similarity for the embedding for the target chemical object and an embedding stored in the vector database.

4. The method of claim 1, wherein inputting the chemical structure information into the trained neural network comprises inputting the chemical structure information into a graph neural network (GNN).

5. The method of claim 1, wherein receiving the query comprising the chemical structure information comprises receiving one or more of XYZ data for the target chemical object or a text string representation of the target chemical object.

6. The method of claim 1, further comprising
receiving labeled training data for a plurality of chemical objects, the labeled training data comprising a property for each chemical object of the plurality of chemical objects, and
further training the neural network to predict the property.

7. The method of claim 6, further comprising, after further training the neural network, updating the vector database by using the neural network to determine, for each chemical object of a plurality of chemical objects in the vector database, a predicted value for the property.

8. The method of claim 7, wherein
updating the vector database is performed prior to the receiving the query,
receiving the query comprises receiving property information, and
retrieving query results from the vector database is further based on the property information and the predicted values for the property for corresponding chemical objects of the query results.

9. The method of claim 7, wherein updating the vector database comprises forming a user version of the vector database.

10. The method of claim 1, wherein the chemical structure information for the target chemical object comprises one or more of structural information for a molecule or structural information for a solid state material.

11. A method of forming a vector database using a neural network, the method comprising:
inputting a set of training data into the neural network, the set of training data comprising structural information for each chemical object of a plurality of chemical objects;
training the neural network; and
using the trained neural network to form the vector database by generating embeddings of chemical structures, each embedding comprising a vector representation of a chemical structure, and storing the embeddings with metadata in the vector database.

12. The method of claim 11, wherein training the neural network comprises using unsupervised training, wherein the unsupervised training comprises masking one or more atoms in a molecule of a training data set and training the neural network to predict the location of the one or more atoms.

13. The method of claim 11, wherein training the neural network comprises using supervised training.

14. The method of claim 13, wherein the supervised training comprises inputting, for each chemical object of the plurality of chemical objects, chemical property information comprising an energy, and wherein training the neural network comprises training the neural network to predict energy.

15. The method of claim 11, wherein training the neural network comprises training a graph neural network.

16. A computing system implementing a neural network, the computing system comprising:
a logic subsystem; and
a storage subsystem comprising
a vector database comprising
a plurality of embeddings of a corresponding plurality of chemical objects, each embedding comprising a vector representation of a chemical structure for a corresponding chemical object, and
metadata comprising chemical object identifications corresponding to the plurality of embeddings, and
instructions executable by the logic subsystem to
receive a query comprising chemical structure information for a target chemical object,
based on the query, input the chemical structure information into the neural network, the neural network configured to produce embeddings of chemical structures,
receive, from the trained neural network, an embedding comprising a vector representation of the target chemical object,
based on the vector representation of the target chemical object, retrieve query results from the vector database, the query results comprising a set of embeddings of chemical objects, and
output the query results.

17. The computing system of claim 16, wherein the neural network comprises a graph neural network (GNN).

18. The computing system of claim 16, wherein the instructions executable to receive the query comprise instructions executable to receive one or more of XYZ data for the target chemical object or a text string representation of the target chemical object.

19. The computing system of claim 16, wherein the instructions are further executable to
receive labeled training data for a set of chemical objects, the labeled training data comprising property information for a property for each chemical object of the set of chemical objects, and
train the neural network to predict the property.

20. The computing system of claim 19, wherein the instructions are further executable to, after training the neural network, update the vector database by using the neural network to, for each chemical object of the plurality of chemical objects in the vector database,
determine a predicted property value for the chemical object, and
store the predicted property value in the vector database.
